(19) United States
(12) Patent Application Publication
(10) Pub. No.: US 2006/0206009 A1
(43) Pub. Date: Sep. 14, 2006

(54) Movable table mounted split access port with removable handle for minimally invasive surgical procedures

(75) Inventors: Ronald S. Von Wald, Centerville, MN (US); Todd W. Sharratt, Stillwater, MN (US); Eric N. Fraser, Vadnais Heights, MN (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: The LeVahn Intellectual Property Holding Company, LLC, St. Paul, MN (US)

(21) Appl. No.: 11/351,890

(22) Filed: Feb. 10, 2006

Related U.S. Application Data
(60) Provisional application No. 60/720,834, filed on Sep. 27, 2005.
(60) Provisional application No. 60/651,717, filed on Feb. 10, 2005.

(51) Int. Cl. A61B 1/32

(52) U.S. Cl. 600/231

(57) ABSTRACT

A port for providing access to a surgical site in a patient's body includes a plurality of segments that when positioned substantially adjacent to each other form a substantially uninterrupted outer perimeter and an internal access portal to the surgical site The port also includes a locking mechanism for retaining the plurality of segments substantially adjacent to each other.

### CROSS REFERENCE TO RELATED APPLICATIONS

The present application claims priority of U.S. Provisional Application Ser. No. 60/651,717 which was filed on Feb. 10, 2005 and is incorporated by reference in its entirety. The present application also claims priority of U.S. Provisional Application Ser. No. 60/720,834 which was filed on Sep. 27, 2005 and is incorporated by reference in its entirety.

### BACKGROUND OF THE INVENTION

The present invention relates to an apparatus that provides access to a surgical site. In particular, the present invention relates to a table mounted apparatus that provides minimally invasive access to a surgical site on an area of the body such as a spine.

The spine includes a number of vertebrae separated by compressible discs that are aligned generally in a column. The vertebrae and the discs have internal through bores that align to form a spinal cavity. Nerves pass through the spinal cavity and provide feeling throughout the body by providing impulses to the brain. Besides providing a passage for the nerves to connect to the brain, the vertebrae and discs provide protection for the nerves.

When the vertebrae, discs or the muscle supporting the spinal column are damaged, a surgical procedure may be required to correct the damage. Because of the sensitive nature of the spine, minimally invasive procedures are being used to minimize the trauma to the spine and also minimize the likelihood of permanent neural damage caused by the surgical procedure. By minimally invasive is meant a surgical procedure where the amount of trauma caused by the surgical procedure is minimized.

Minimally invasive surgical techniques may include the use of retractors that are designed to retract flesh from an incision of a minimized length. Besides the use of an incision, a surgical site can be accessed through the use of a series of dilators and a surgical access port. The dilators create an opening in the skin by gradually forcing the flesh from the surgical site by inserting larger diameter dilators one over another. The access port is positioned over the largest dilator and into the flesh thereby allowing the dilators to be removed. With the dilators removed from the body, the surgical procedure is performed through the access port.

### SUMMARY OF THE INVENTION

The present invention includes a port for providing access to a surgical site in a patient's body. The port includes a plurality of segments that when positioned substantially adjacent to each other form a substantially uninterrupted outer perimeter and an internal access portal to the surgical site. The port also includes a locking mechanism for retaining the plurality of segments substantially adjacent to each other.

### BRIEF DESCRIPTION OF THE DRAWINGS

FIG. 1a is a perspective view of a split access port of the present invention being positioned proximate a surgical site and above a plurality of dilators.

FIG. 1b is a perspective view of the split access port of the present invention being positioned into the surgical site about the plurality of dilators.

FIG. 1c is a perspective view of the split access port of the present invention providing access to the surgical site by inserting the split access port into the patient and removing the plurality of dilators.

FIG. 2 is a perspective view of the split access port of the present invention mounted to a table mounted support apparatus with retractor blade holders.

FIG. 3 is an exploded view of the split access port of the present invention.

FIG. 4 is a perspective view of the split access port of the present invention being utilized to enlarge the access to the surgical site by retracting segments of the split access port from each other.

FIG. 5 is a perspective view of the split access port of the present invention having distal ends rotated away from the surgical site to enlarge the access to the surgical site.

FIG. 6 is a perspective of another embodiment of the split access port of the present invention having four segments.

FIG. 7 is a perspective view of another embodiment of the split access port of the present invention having two segments.

FIG. 8 is a perspective view of another embodiment of the split access port of the present invention having two segments positioned apart from each other.

### DETAILED DESCRIPTION OF THE PREFERRED EMBODIMENTS

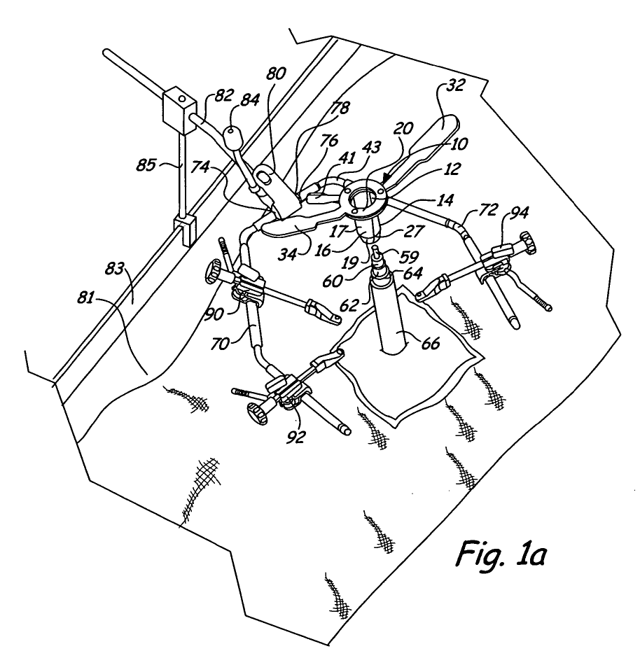

A table mounted split access port is generally illustrated at 10 in FIG. 1. The table mounted split access port 10 is utilized to gain access to the surgical site. The surgical site typically is the spine where the split access port 10 can approach the spine from a posterior or anterior direction. However the split port 10 can also be used to access other surgical sites in the body.

The split port 10 typically includes three segments 12, 14 and 16 that complete a substantially circular perimeter 17. A split access port 10 having configurations other than a substantially circular perimeter are within the scope of the present invention including a split access port with an ellipsoidal perimeter.

Typically the segments 12, 14 and 16 have outer perimeters with a substantially 1200 arc such that the three segments 12, 14 and 16 complete the substantially circular perimeter 17. However, a split access port 10 with more than two segments is within the scope of the present invention, including a split access port 10 with four or five segments. A split port with segments with arcuate outer surfaces of different lengths are also within the scope of the present invention, provided the segments typically complete a continuous perimeter.

The split access port 10 is preferably constructed of a radio-lucent material such as aluminum or an engineering polymer. What is meant by radio-lucent is a material that is partially or wholly permeable to radiation such as an X-ray. By engineering polymer is meant a polymer that is useful as a material of construction of a part or component of the device. However, nonradio-lucent materials of construction are also within the scope of the present invention.

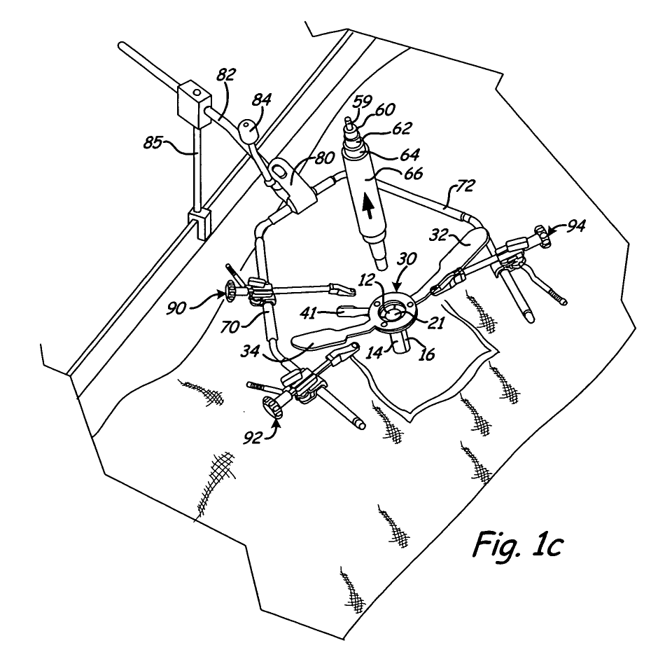

Referring to FIGS. 3-5, each of the segments 12, 14 and 16 includes an arcuate blade portion 18 having an inner arcuate surface 20 and an outer arcuate surface 22. Each segment 12, 14 and 16 also includes a horizontal portion 23 extending from a proximal end of the blade portion 18.

A peg 24 is attached to the horizontal portion 23 by engaging an annular channel 26 with a slot (not shown) in the horizontal portion 23. However, the peg 24 may also be attached to the substantially horizontal portion 24 by other mechanisms including a weld, a threaded engagement, a frictional engagement or an adhesive.

Each segment 12, 14 and 16 includes a slot 13 on a first side surface that accepts an extension 15 on a side surface of the adjacent section to retain the sections together. However, segments 12, 14 and 16 with smooth side surfaces are within the scope of the scope of the present invention.

A handle 30, having gripping ends 32, 34, attaches to the segments 12, 14 and 16 by engaging annular grooves 25 in the pegs 24. The handle 30 includes a substantially circular ring 36 from which the gripping ends 32, 34 extend. The generally circular ring 36 includes three apertures 38 that are spaced apart for accepting the three pegs 24. However the number of aperture 38 in the ring 36 will vary with the number of segments and pegs used to define the access port 10.

A locking plate 40 rotatably attaches to the substantially circular ring 36 by positioning pins 50 through a first set of slots 42 in the locking plate 40. The pins 50 frictionally engage apertures 37 in the substantially circular ring 36. The frictional engagement of the pins 50 with the apertures 37 retains the locking plate 40 to the handle 30 and wherein the locking plate 40 rotatably moves an arcuate distance defined by a length of the slots 42.

When one end of the first set of slots 42 are positioned proximate the pins 50, an elongated end 44 of a second set of retaining slots 45 aligns with the apertures 38 such that the pegs 24 are position through the elongated ends 44 and the apertures 38. Typically, the locking plate 40 includes three retaining slots 45 corresponding to the number of segments 12, 14 and 16 and peg 24 extending therefrom. However, the number of retaining slots 45 will vary with the number of segments and pegs utilized to define the access port 10.

As the locking plate 40 rotates by applying manual force to a handle 41, the retaining slots 45 engage the annular groove 25 in each peg 24. Side surfaces 47, 48 of the retaining slots 45 engage the annular groove 25 in the peg 24 and exert a force away from the surgical site which in turn forces distal ends 27 of the blade portions 18 of the segments 12, 14 and 16 toward each other to retain the port 10 in the typical configuration with the uninterrupted substantially circular perimeter 17.

A distance between the side surface 47, 48 and a diameter of a retaining end 46 is less than the diameter of the peg 24 at a top end such that the side surfaces 47, 48 and the retaining end 46 of the retaining slots 45 slidably engage the annular groove 25 in the peg 24 to retain the sections 12, 14 and 16 of the access port 10 in a selected configuration.

Referring to FIGS. 1a-1c, the surgical site is prepared by forcing a wire 59 through the skin and toward the spine. A typical wire 59 is a “K” wire that has a diamater of about forty thousandths of an inch. The spine is typically X-rayed to verify the wire is positioned in the selected position.

With the wire 59 in the selected position, a series of dilators 60, 62, 64 and 66 are inserted into the body of the patient to expand the skin and flesh to expose the surgical site while minimizing trauma to the patient. The first dilator 60 is positioned about the wire (not shown) to retract the skin and the flesh. Progressively larger diameter dilators 62, 64 and 66 are positioned about the smaller dilators to retract the skin and flesh from the surgical site. Each of the dilators 60, 62, 64 and 66 has a tapered end to aid in inserting the dilator into the skin and flesh. While four dilators 60, 62, 64 and 66 are typical, utilizing one or more dilators are within the scope: of the present invention.

With the skin and flesh dilated to a selected diameter, the split access port 10 is positioned about the largest diameter dilator 66 and into the body at the surgical site. The split access port 10 positioned over the dilator 66 such that the inner arcuate surfaces 20 of the segments 12, 14 and 16 are proximate the dilator 66 and the outer arcuate surfaces 22 engage the flesh to gain access to the surgical site. The distal ends 27 of the blade portions 18 of the segments 12, 14 and 16 are typically tapered to a narrow edge to aid in inserting the sections 12, 14 and 16 into the flesh.

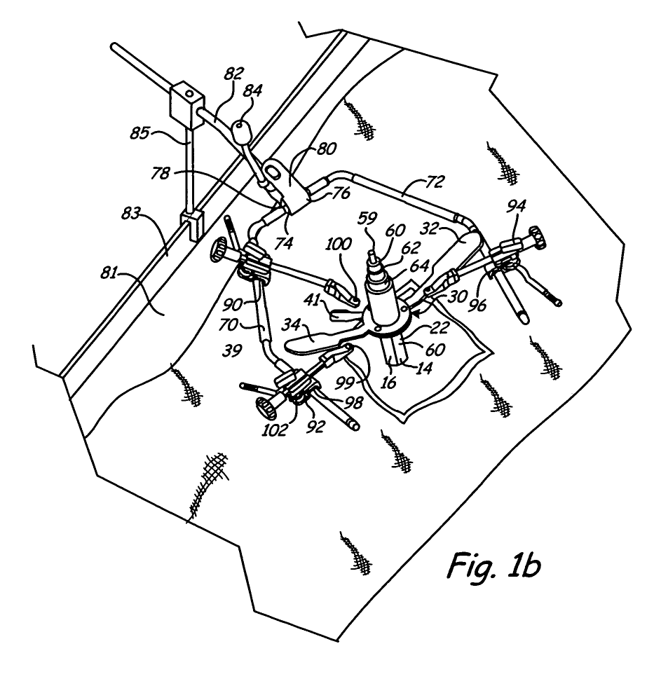

With the split access port 10 positioned over the dialator 66 the surgeon or other operating personnel grips the gripping ends 32, 34 to manually force the split access port 10 into the surgical site and to manipulate the position of the split access port 10 within the patient. With the split access port 10 in the selected position in the surgical site, the dilators 60, 62, 64 and 66 are removed with manual force to provide access to the surgical site through the access port 10 defined by the inner arcuate surfaces 20 of the blade portions 18 as best illustrated in FIGS. 1c and 2.

Having access to the surgical site, the surgeon rotates a handle 41 on the locking plate 40 in the direction of arrows 43 such that the elongated ends 44 of the retaining slots 45 align with the apertures 38. With the elongated ends 44 aligned with the apertures 38, the side surfaces 47, 48 and the engaging end 46 of the retaining slot 45 disengage the annular grooves 27 in the pegs 24. Manual force is placed upon the handle 30 to remove the handle 30 including the locking plate 40 from the pegs 24.

With the split access port 10 positioned in the selected position within the body at the surgical site, support arms 70, 72 are positioned about the surgical site. The support arms 70, 72 have pivot balls 74, 76 that are rotatably positionable within a clamping bore 78 of a clamp 80 that is secured to a surgical table 81 by engaging a support rod 82 extending from and rotatably attached to the clamp 80. The support rod 82 is mounted to a rail 83 of the surgical table 81 in a with a retractor support apparatus 85 that is well known in the art and is described in U.S. Pat. Nos. 4,617,916, 4,718,151, 4,949,707, 5,400,772, 5,741,210, 6,042,541, 6,264,396 and 6,315,718 all of which are herein incorporated by reference.

With the support arms 70, 72 in selected positions, a lever 84 is rotated which causes the clamping bore 78 to constrict and secure the pivot balls 74, 76 in selected positions within the clamping bore 78. The support arms 70 and 72 are independently adjustable into an infinite number of selected positions through use of a clamp 80 which is described in U.S. Pat. Nos. 5,899,627 and 6,264,396, both of which are herein incorporated by reference in their entireties.

Referring to FIGS. 1b, 1c and 2, with the support arms 70, 72 secured in the selected positions, articulated retractor blade holders 90, 92 and 94 are positioned on the support arms 70, 72, respectively. Throughout the application like components of the retractor blade holders 90, 92 and 94 will be given like reference characters.

The retractor blade holders 90, 92 and 94 have a clamp 96 with a clamping socket 98 that is manually positioned in selected positions about support arms 70, 72. The clamps 96 are slidably positionable on the support arms 70, 72 to position a distal end 99 proximate the pegs 24 extending from the substantially horizontal portions 23 on the segments 12, 14 and 16.

The segments 12, 14 and 16 are secured to the retractor blade holders 90, 92 and 94, respectively, by inserting the pegs 24 into apertures 100 at the distal ends 99 of the articulated retractor holders 90, 92 and 94. Spring load balls (not shown)retained within the distal ends 99 engage the annular grooves 25 about the pegs 24 to rotatably retain the segments 12, 14 and 16 to the retractor holders 90, 92 and 94, respectively.

With the segments 12, 14 and 16 attached to the articulated retractor holders 90, 92 and 94, respectively, actuating mechanisms 102 are rotated by moving handles 104 which cause the clamping sockets 98 to constrict and secure the articulated retractor handles 90, 92 and 94 in selected positions with a frictional engagement.

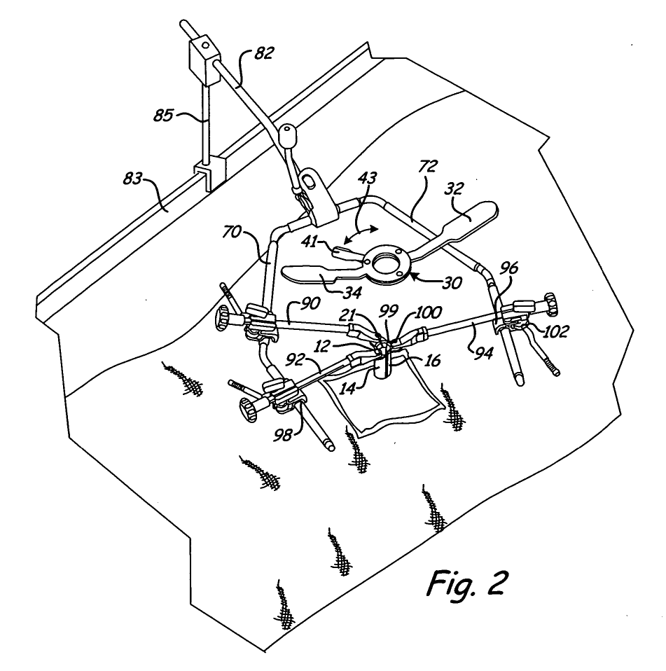

With the segments 12, 14 and 16 secured to the support arms 70, 72, the surgeon can perform the surgical procedure through a generally circular access portal 21 as illustrated in FIG. 2. Referring to FIG. 4, the surgeon can manipulate a rack and pinion systems 110 on the articulated retractor handle 118 to separate the segments 12, 14 and 16, respectively, in direction of arrows 119 and thereby increase the area of access to the surgical site. The rack and pinion system 110 is manipulated by rotating a gear pin 112 in a gear box 114 having cogs (not shown) that engage a rack 116 on the handle 118. A pawl 120 engages the rack 116 and prevents the segments 12, 14 and 16 from moving toward each other as a force is applied to the segments 12, 14 and 16 by the retracted flesh.

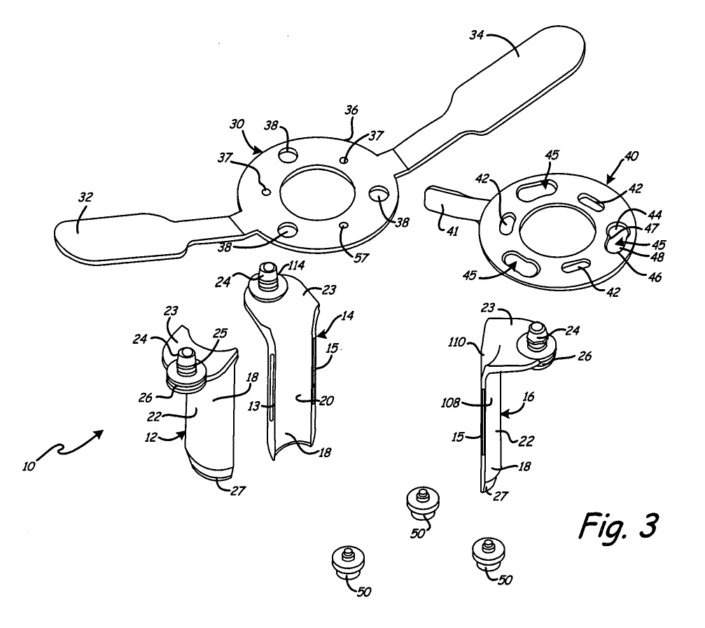

Referring to FIG. 5, access to the surgical site can be enlarged by manipulating an articulated joint 130 at the distal ends of the retractor holders 90, 92 and 94 which increase a distance between the ends 17 of the segments 12, 14 and 16, respectively. The articulated joint 130 is manipulated by rotating a knob 132 at a proximal end of the retractor handle 118. The knob 132 threadably engages a threaded region at the proximal end such that the knob 132 moves along a length of the retractor handle 118.

A push rod (not shown) is non-rotatably attached to the knob 132 and has a spherical end (not shown) positioned within a vertical slot 136 within a blade holder 138. As the push rod is moved, the spherical end engages the slot 136 which causes the blade holder 138 to pivot about a pivot pin 140. The pivotal engagement of the blade holder 138 and the handle 118 define the articulated joint that causes the distal ends 27 of the segments 12, 14 and 16 to be further separated or drawn toward each other, in a direction of arrows 141 depending upon the amount of access required for the surgical procedure.

In use, the surgical site is prepared by forcing the wire 59 through the skin and toward the spine. The spine is X-rayed to verify the wire is positioned in the selected position.

With the wire 59 in the selected position, the series of dilators 60, 62, 64 and 66 are employed to expand the skin and flesh to expose the surgical site. The first dilator 60 is positioned about the wire 59 to retract the skin and the flesh. Progressively larger diameter dilators are positioned about the smaller dilators to retract the skin and flesh from the surgical site. Each of the dilators has a tapered end to aid in inserting the dilator into the skin and flesh.

With the skin and flesh dilated to a selected diameter, the split access port 10 is positioned about the largest dilator 66 and into the body. Manual force is applied to the split access port 10, typically by gripping the handles 30, 32, to position the split access port 10 within the body at the surgical site.

With the access port 10 positioned within the body, the support arms 70, 72 are positioned in selected positions about the surgical site and the clamp 80 is positioned into the clamping position to secure the support arms 70, 72 in the selected positions.

The articulated retractor handles 90, 92 and 94 are positioned on the support arms 70, 72 by forcing the clamping sockets 98 about the support arms 70, 72. The articulated retractor handles 90, 92 and 94 are slidably movable into selected positions on the support arms 70, 72 and are secured in the selected positions by rotating the actuating mechanisms 102 which causes the clamping sockets 98 to constrict and frictionally secure the articulated retractor handles 90, 94 and 94 to the support arms 70, 72.

The distal ends 19 of the articulated retractor handles 90, 92 and 94 are positioned proximate the pegs 24 extending from the segments 12, 14 and 16 by manipulating the rack and pinion mechanism 110 and the articulated joint 130. The pegs 24 are inserted into the apertures 100 proximate the distal ends 99 of retractor handles 90, 92 and 94 and are rotatably retained therein.

As illustrated in FIG. 2, after the dilators 60, 62, 64 and 66 are removed, the surgeon can perform a surgical procedure through the substantially circular cross-section access portal 21 as illustrated in FIG. 2. The surgeon can also manipulate the rack and pinion mechanism 110 by manipulating the gear pin 112 to force the segments 12, 14 and 16 apart in direction of arrows 119 to expand the access to the surgical site as best illustrated in FIG. 4. Other mechanisms that spread the segments 12, 14 and 16 apart are also within the scope of the present invention including a setscrew.

The surgeon may increase the access to the surgical site by manipulating the articulated joint 130 by rotating the knob 132 to force the push rod (not shown) to move within the channel 136 in the blade holder 138. As the push rod moves within the channel 136, the blade holder 138 pivots about the pivot pin 140 which forces the ends 27 of the segments 12, 14 and 16 apart in the direction of arrows 141 and increases the access to the surgical site as illustrated in FIG. 5.

With the segments 12, 14 and 16 secured in the selected positions, the surgeon performs the minimally invasive surgery through the access portal 21, whether substantially circular, elongated or elongated and angled. When the surgery is completed the segments 12, 14 and 16 are positioned proximate each other by manipulating the rack and pinion mechanism 110 and the articulated joint 130. The pegs 24 are removed from the articulated retractor holders 90, 92 and 94 with manual force and the segments 12, 14 and 16, are removed the surgical site, thereby allowing the wound to be closed. Depending upon the length of the segments 12, 14 and 16 the surgery upon the spine can be performed with an anterior or posterior approach. Also, the split access port 10 of the present invention may also be used to provide access to other surgical sites within the body besides the spine.

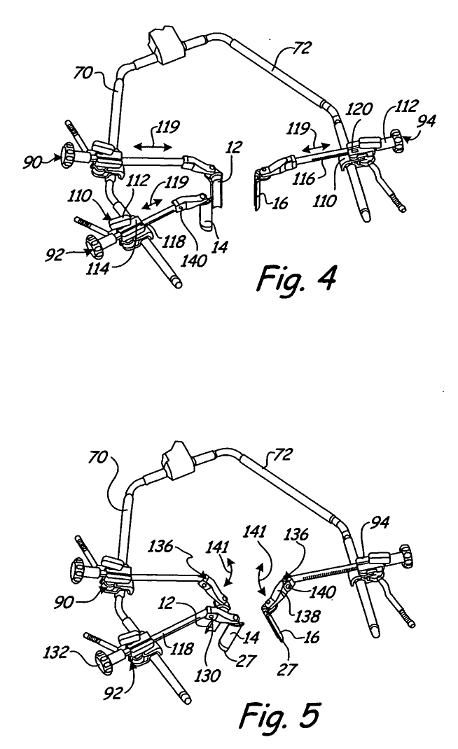

Referring to FIG. 6 another embodiment of the split access port is generally illustrated at 210. The access port 210 includes four segments 212, 214, 216 and 218 of substantially the same configuration and having an arcuate length of about 90 degrees such that the four segments 212, 214, 216 and 218 complete a substantially cylindrical perimeter 220 and access port 222.

The segments 212, 214, 216 and 218 are retained to a handle 224 having four slots (not shown) to retain pegs 226 having the same configuration as the peg 24 in a manner as described with respect to the embodiment 10. The four segment access port 210 allows for greater access to the surgical site when compared to the embodiment 10.

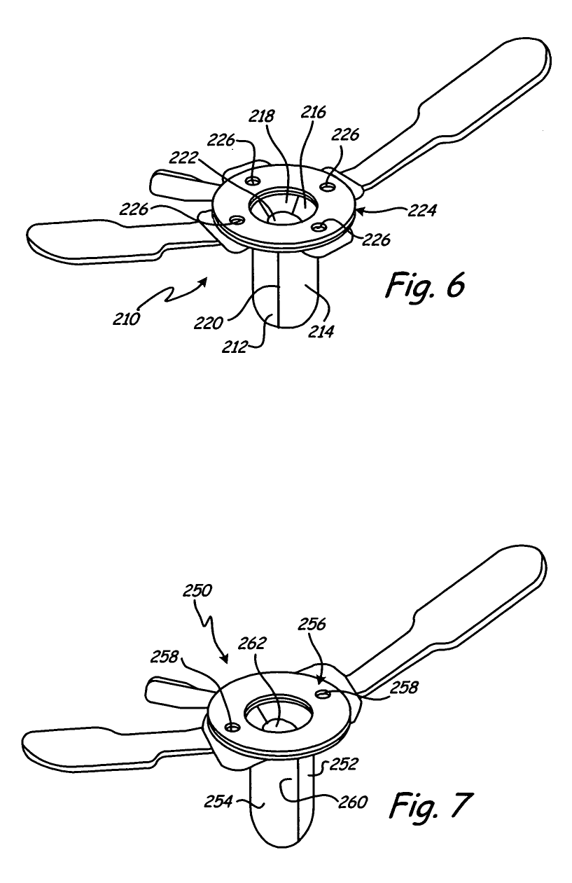

Referring to FIG. 7 another embodiment of the split access port is generally illustrated at 250. The access port 250 includes two segments 252 and 254 of substantially the same configuration and having an arcuate length of about 180° such that the two segments 252 and 254 complete a substantially cylindrical perimeter 260 and access port 262.

The segments 252 and 254 are retained to a handle 256 having two slots (not shown) to retain pegs 258 having the same configuration as the peg 24 in the same manner as described with respect to the embodiment 10. The two segment access port 250 allows adequate flexibility in manipulating the access to the surgical site while still being capable of providing access to the surgical site when compared to the embodiment 10.

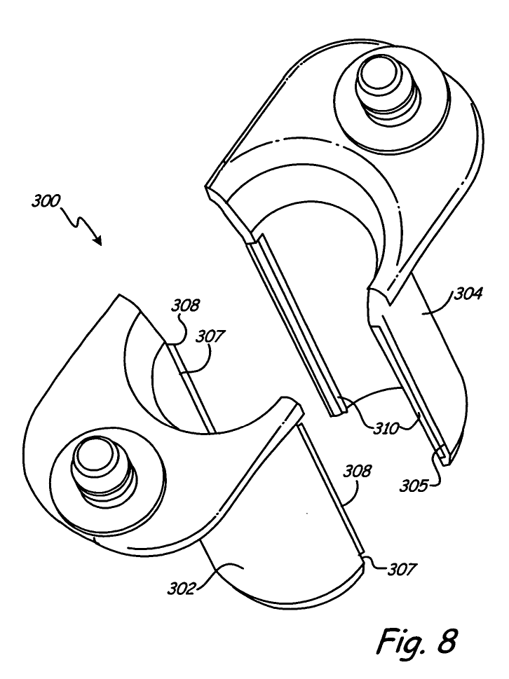

Another embodiment of the split access port is generally illustrated in FIG. 8 at 300. The split access port 300 includes left and right halves 302, 304. The left and right halves 302, 304 have mating surfaces to prevent the halves from separating as force is applied to the split access port 300 while being inserted into the body without utilizing a handle. The left half 302 includes raised ridges 308 along surfaces 307 that abut surfaces 305 on the right half 304. The raised ridges 308 are positioned within cut out channels 310 along the surface 305 where the raised ridge 308 engages the surfaces defining the cut out channels 310 to retain the left and right halves 302, 304 together when manually forced into the surgical wound. Once positioned within the surgical wound, the left and right halves 302, 304 are attached to the retractor support arms 70, 72 with retractor handles.

Although the present invention has been described with reference to preferred embodiments, workers skilled in the art will recognize that changes may be made in form and detail without departing from the spirit and scope of the invention.

What is claimed is:

1. A port for providing access to a surgical site in a patient's body comprising:
a plurality of segments that when positioned substantially adjacent to each other form a substantially uninterrupted outer perimeter and an internal access portal to the surgical site; and
a locking mechanism for retaining the plurality of segments substantially adjacent to each other.

2. The port of claim 1 and wherein each segment comprises a peg that is engaged by the locking mechanism to retain the plurality of segments substantially adjacent to each other.

3. The port of claim 2 and wherein the locking mechanism comprises:
a first layer having a plurality of apertures having a size sufficient to pass the plurality of pegs therethrough; and
a second layer rotatably attached to the first layer and having a plurality of arcuate slots having an elongated end having a size sufficient to pass the plurality of pegs therethrough and a narrow end that engages the pegs and retains the pegs to the locking mechanism.

4. The port of claim 1 and wherein the locking mechanism comprises at least one handle for manually forcing the segments into the surgical site.

5. The port of claim 1 and wherein each segment comprises:
an arcuate blade portion having a proximal end and a tapered distal end; and
a portion extending from the proximal end of the arcuate blade portion.

6. The port of claim 5 and wherein each segment further comprises a peg extending from the portion for engaging the locking mechanism.

7. The port of claim 1 and wherein the plurality of segments comprises three segments.

8. The port of claim 1 and wherein the plurality of segments comprises four segments.

9. An apparatus for providing access to a surgical site comprising:
a surgical table having a rail;
a retractor support apparatus attached to the rail;
at least one retractor support arm attached to the retractor support apparatus and positionable proximate the surgical site;
an access port comprising:
a plurality of segments that when positioned substantially adjacent to each other form a substantially uninterrupted outer perimeter and an internal access portal to the surgical site; and
a locking mechanism for retaining the plurality of segments substantially adjacent to each other and wherein the locking mechanism is removed from the surgical site after the plurality of segment provide access to the surgical site; and
a plurality of retractor blade holders for attaching the plurality of segments to the at least one retractor support arm.

10. The apparatus of claim 9 and wherein each segment comprises a peg that is engaged by the locking mechanism to retain the plurality of segments substantially adjacent to each other.

11. The apparatus of claim 10 and wherein the locking mechanism comprises:
a first layer having a plurality of apertures having a size sufficient to pass the plurality of pegs therethrough; and
a second layer rotatably attached to the first layer and having a plurality of arcuate slots having an elongated end having a size sufficient to pass the plurality of pegs therethrough and a narrow end that engages the pegs and retains the pegs to the locking mechanism.

12. The apparatus of claim 9 and wherein the locking mechanism comprises at least one handle for manually forcing the segments into the surgical site.

13. The apparatus of claim 9 and wherein each segment comprises:
an arcuate blade portion having a proximal end and a tapered distal end;
a portion extending from the proximal end of the arcuate blade portion; and
a peg extending from the portion for engagement with the locking mechanism.

14. The apparatus of claim 9 and each retractor holder comprises:
a rack and pinion system for laterally moving the segments to provide access to the surgical site; and
an articulated joint for rotatably moving each segment to provide access the surgical site.

15. The apparatus of claim 9 and wherein the at least one retractor support arm comprises:
a clamp attached to the retractor support apparatus and having a clamping bore therethrough;
a left support arm having a left pivot ball attached thereto and positioned within the clamping bore; and
a right support arm having a right pivot ball attached thereto and positioned with the clamping bore and wherein the left and right support arms independently move with respect to each other into selected positions and are retained in the selected positions by positioning the clamp into the clamping position which constricts the clamping bore into a frictional engagement with the left and right pivot balls.

16. A method of providing access to a surgical site using minimally invasive techniques comprising:
positioning a patient on a surgical table;
inserting a plurality of dilators of increasing diameter into the patient to retract skin and flesh from the surgical site;
providing an access port with a plurality of segments in selected positions that define an interior access portal and have a substantially uninterrupted outer perimeter and a locking mechanism for retaining the plurality of segments in the selected positions;
manually positioning the access port about an outer dilator and manually forcing the access port into the skin and flesh proximate the surgical site;
removing the plurality of dilators from the surgical site such that the surgical site is accessible through the interior access portal;
attaching a retractor support apparatus to the surgical table;
attaching at least one retractor support arm to the retractor support apparatus and positioning the at least one retractor support arm in a selected position proximate the surgical site;
detaching the locking mechanism from the plurality of segments; and
attaching a plurality of retractor handles to both the retractor support arm and the plurality of segments wherein one segment is attached to each retractor handle such that each segment is attached to the table mounted retractor support arm.

17. The method of claim 16 and further comprising manipulating at least one retractor handle to adjust a lateral position of at least one segment.

18. The method of claim 17 and further comprising manipulating at least one retractor handle to adjust an angular position of at least one segment.

19. The method of claim 16 and further comprising inserting a wire into the body proximate the surgical site and positioning a dilator about the wire.

20. The method of claim 16 and further comprising removing the segments from the surgical upon completion of the surgical procedure.
